(19) United States
(12) Patent Application Publication
(10) Pub. No.: US 2025/0387132 A1
(43) Pub. Date: Dec. 25, 2025

(54) MEDICAL DEVICES AND RELATED SYSTEMS AND METHODS OF USE

(71) Applicant: Boston Scientific Medical Device Limited, Galway (IE)

(72) Inventors: Yogesh Kishor VIKHARANKAR, Pune (IN); Hitendra PUROHIT, Pune (IN); Aditya DHANOTIYA, Indore (IN); Sucheta MISHRA, Pune (IN); Snehal PATIL, Pune (IN)

(73) Assignee: Boston Scientific Medical Device Limited, Galway (IE)

(21) Appl. No.: 19/242,086

(22) Filed: Jun. 18, 2025

Related U.S. Application Data
(60) Provisional application No. 63/662,464, filed on Jun. 21, 2024.

(51) Int. Cl. A61B 17/221; A61B 10/02; A61B 18/00; A61B 18/14

(52) U.S. Cl. CPC A61B 17/221; A61B 18/1477; A61B 10/0233; A61B 2017/2212; A61B 2017/2215; A61B 2018/00077; A61B 2018/00577; A61B 2018/00595; A61B 2018/00601; A61B 2018/00982

(57) ABSTRACT

This disclosure includes a medical device comprising a handle assembly including a first actuator; a shaft extending distally from the handle assembly; a first jaw member movably coupled to a distal portion of the shaft; a second jaw member movably coupled to the distal portion of the shaft and to the first jaw member; and a needle positioned between the first jaw member and the second jaw member. The needle may be configured to receive electrical energy to facilitate cutting, ablating, and/or cauterizing tissue.

### CROSS-REFERENCE TO RELATED APPLICATIONS

This application claims the benefit of priority from U.S. Provisional Application No. 63/662,464, filed on Jun. 21, 2024, which is incorporated by reference herein in its entirety.

### TECHNICAL FIELD

Various aspects of this disclosure relate generally to medical devices, systems, and procedures. Particular aspects relate to medical devices and systems for removing material, e.g., obstructions, from one or more body lumens and/or passageways, among other aspects.

### BACKGROUND

During noninvasive procedures, a physician may use a capture device (e.g., a basket or grasper) to engage objects in a body. For example, in some urology procedures, the physician may advance a sheath into a kidney, extend a basket from the sheath, engage a stone in the kidney with the basket, and retract the basket to capture the stone. Often, a capture device may be deployed through a working channel of a ureteroscope, an endoscope, or other insertable medical device. For example, a ureteroscope may be placed in the ureter or kidney, and a capture device may be deployed through a working channel of the ureteroscope and used to “grab” a stone or other tissue for removal. A plurality of stones and/or stone fragments may need to be removed during the procedure, requiring the basket to be extended numerous times. Laser lithotripsy may be used to break apart larger stones but may increase risk to the patient. For example, use of a laser may increase the chances of damage to the inner linings of a body lumen due to heat generation and/or touching the inner linings of a body lumen.

### SUMMARY

The present disclosure relates to, among other things, medical devices and systems for removing obstructions from body lumens and/or passageways, and related methods of use. Aspects of this disclosure may decrease the time to remove an obstruction, such as a kidney stone, from a patient's body. Further, for example, aspects of the present disclosure may reduce the level of skill of a practitioner sufficient to complete such obstruction removal procedure, and/or may prevent damage to one or more devices during a procedure. Aspects of this disclosure may facilitate preventing damage to a patient during a medical procedure, such as injury associated with laser lithotripsy. Although primarily discussed in the context of kidney stone removal procedures, any of the systems, devices, and features thereof discussed herein may be used or adapted for use in other parts of the body to remove other materials or obstructions. The medical devices and systems disclosed herein in various examples may include one or more of the features or components described in connection with any of the other examples.

This disclosure includes a medical device comprising a handle assembly including a first actuator; a shaft extending distally from the handle assembly; a first jaw member movably coupled to a distal portion of the shaft; a second jaw member movably coupled to the distal portion of the shaft and to the first jaw member; and a needle positioned between the first jaw member and the second jaw member. The needle may be configured to receive electrical energy to facilitate cutting, ablating, and/or cauterizing tissue.

In some aspects, the medical device may include one or more of the following features. The first jaw member may include a first basket portion including a first recessed portion; and the second jaw member may include a second basket portion including a second recessed portion. Each of the first basket portion and the second basket portion may include a plurality of apertures. Each of the first basket portion and the second basket portion may include a polymer coating. The first actuator may be configured to transition the first jaw member and the second jaw member between an open configuration in which the first jaw member and the second jaw member are spaced from each other and a closed configuration in which the first basket portion and the second basket portion form an enclosure. The handle assembly may further comprise a second actuator configured to move the needle relative to the first jaw member and the second jaw member. The first actuator may include a recess configured to receive the second actuator.

In some aspects, the medical device may include one or more of the following features. The needle may include a lumen configured to receive a fluid. The needle may be electrically conductive. A distalmost portion of the needle may include a plurality of sharp tips. The handle assembly may further comprise a motor configured to rotate the needle about a longitudinal axis of the needle. The first basket portion and the second basket portion may be flexible. The first basket portion and the second basket portion may be heat resistant. The first basket portion may include a first oval opening and the second basket portion may include a second oval opening, and the first oval opening and the second oval opening may be substantially the same size and configured to align with each other when the first jaw member and the second jaw member are in a closed configuration. When the first jaw member and the second jaw member are in a closed configuration that forms an enclosure, the first basket portion and the second basket portion may form a proximal opening configured to receive the needle.

This disclosure also includes a medical device comprising a handle assembly including a first actuator and a second actuator; a shaft extending distally from the handle assembly; a first jaw member movably coupled to a distal portion of the shaft; a second jaw member movably coupled to the distal portion of the shaft; and a needle positioned between the first jaw member and the second jaw member. The first actuator may be configured to actuate the first jaw member and the second jaw member to transition between an open configuration and a closed configuration. The second actuator may be configured to move the needle relative to the first jaw member and the second jaw member.

In some aspects, the medical device may include one or more of the following features. The needle may be electrically conductive. The first jaw member may include a first basket portion and the second jaw member may include a second basket portion, and when the first jaw member and the second jaw member are in the closed configuration, the first basket portion and the second basket portion form a proximal opening configured to receive the needle.

This disclosure also includes a method comprising positioning a shaft of a medical device at target site of a patient; moving a first jaw member and a second jaw member of the medical device from an open configuration in which the first jaw member and the second jaw member are spaced from each other to a closed configuration in which the first jaw member and the second jaw member form an enclosure; moving a needle of the medical device distally into the enclosure; and applying electrical energy to the needle.

### BRIEF DESCRIPTION OF THE DRAWINGS

The accompanying drawings, which are incorporated in and constitute a part of this application, illustrate exemplary aspects of this disclosure and together with the description, serve to explain the principles of the disclosure.

FIG. 1 is a side view of an exemplary medical device, according to aspects of this disclosure.

FIGS. 2A and 2B are magnified views of a distal portion of the medical device of FIG. 1, according to aspects of this disclosure.

FIG. 3 is a perspective view of a distal portion of another exemplary medical device, according to aspects of this disclosure.

FIG. 4 is a front view of a distal portion of the medical device of FIG. 1, according to aspects of this disclosure.

FIG. 5 is a magnified view of a distal portion of the medical device of FIG. 1, according to aspects of this disclosure.

FIG. 6 is a distal portion of an exemplary needle of a medical device, according to aspects of this disclosure.

FIGS. 7A and 7B are side views of a proximal portion of the medical device of FIG. 1 in different states of operation, according to aspects of this disclosure.

FIG. 8 is a perspective view of a distal portion of the medical device of FIG. 1, according to aspects of this disclosure.

FIG. 9 is a perspective view of a proximal portion of another exemplary medical device, according to aspects of this disclosure.

FIG. 10A is a perspective view of a distal portion of the medical device of FIG. 9, according to aspects of this disclosure.

FIG. 10B is a magnified view of a needle of the medical device of FIG. 9, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, device, or apparatus that comprises a list of elements does not include only those elements, but may include other elements not expressly listed or inherent to such process, method, article, device, or apparatus. The term “exemplary” is used in the sense of “example,” rather than “ideal.” Relative terms such as, for example, “about,” “substantially,” “approximately,” etc., are used to indicate a possible variation of +10% in a stated numeric value or range. The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures.

Although ureteroscopes are referenced herein, reference to ureteroscopes should not be construed as limiting the possible applications of the disclosed aspects. For example, the disclosed aspects may be used with endoscopes, duodenoscopes, bronchoscopes, colonoscopes, catheters, sheaths, diagnostic or therapeutic tools or devices, or other types of medical devices.

Aspects of various devices are now described. Some aspects are described with reference to noninvasive procedures, such as urology procedures, wherein a sheath is advanced to a treatment site, and a capture device is extended from the sheath to engage an object at the treatment site. In urology procedures, for example, the sheath may be inserted into the urethra, moved through the bladder and ureter, and advanced into a calyx of a kidney; and the capture device may be extended distally from the sheath and/or a working channel of a ureteroscope or other medical device to engage one or more stones and/or stone fragments located in the calyx.

References to a particular type of medical procedure, such as a urology procedure; capture device, such as a basket or grasper; organ, such as a kidney; and/or object, such as a stone or stone fragment, are provided for convenience and not intended to limit this disclosure. Accordingly, the concepts described herein may be utilized for any analogous medical device or system.

The medical devices, systems, and methods discussed herein may facilitate visualization of a target site while breaking apart material (e.g. stones, etc.) during a procedure, may reduce the risk of injury to a patient's tissue during a procedure, may reduce the cost of a procedure, may minimize the need for multiple devices during a procedure, and/or may increase accuracy of material removal, among other benefits.

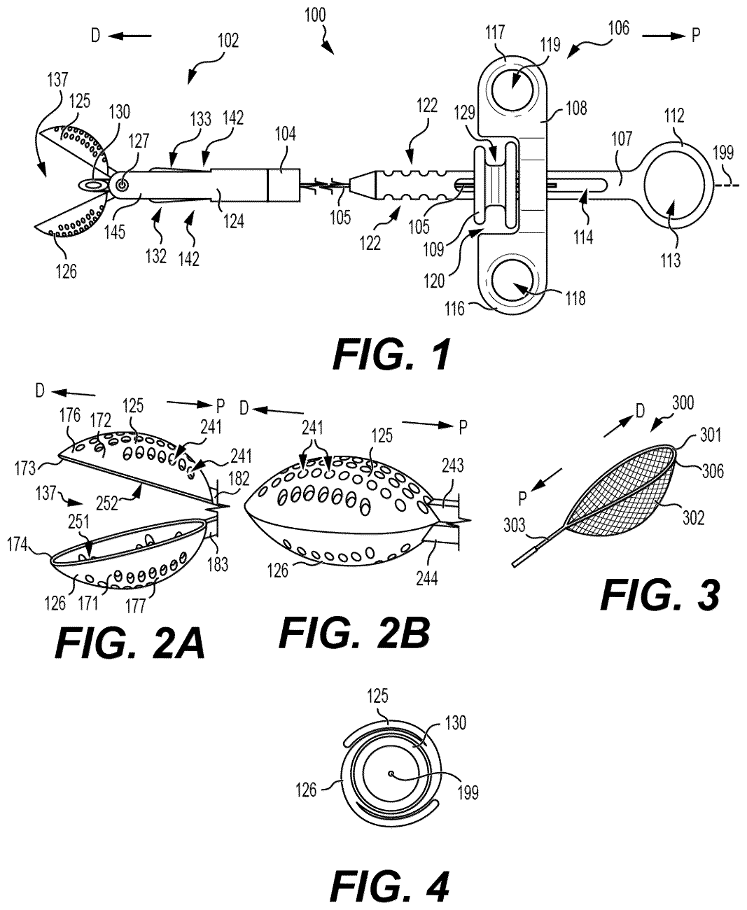

FIG. 1 shows a side view of an exemplary medical device 100 including a handle assembly 106, and a shaft 104 extending distally from the handle assembly 106 to a distal portion 102. Handle assembly 106 may include a handle body 107, a first actuator 108, and a second actuator 109. Handle body 107 may be cylindrical and may include a ring portion 112 at a proximalmost end of handle body 107. Ring portion 112 may form a lumen 113 configured to receive a user's hand, e.g., one or more fingers of the user's hand. In some examples, a slot 114 may extend through handle body 107, and optionally slot 114 may be oval shaped. Grip protrusions 122 may be at a distal portion of handle body 107, and may be configured to facilitate gripping handle body 107. A central, cylindrical passage may extend longitudinally through handle body 107 and may be configured to receive one or more control wires 105 or other members of medical device 100.

First actuator 108 may protrude radially outward, relative to a central longitudinal axis 199 of medical device 100, from handle body 107. First actuator 108 may include a first lumen 118 positioned at a first end 116 of first actuator 108, and a second lumen 119 positioned at a second end 117 of first actuator 108. Each of first lumen 118 and second lumen 119 is configured to receive one or more fingers of a user's hand to facilitate movement of first actuator 108. First actuator 108 may include a central recess 120 configured to receive second actuator 109 (as shown in FIG. 1). First actuator 108 may be moveably coupled to handle body 107 and configured to move proximally and distally relative to handle body 107.

Second actuator 109 may be cylindrical and may include a recess 129 extending circumferentially around second actuator 109. Recess 129 may be configured to receive one or more fingers of a user's hand and to facilitate movement of second actuator 109 relative to first actuator 108 and handle body 107. Second actuator 109 may be coupled to handle body 107 at a position distal from first actuator 108, and may be configured to move proximally and distally relative to handle body 107 and first actuator 108. Each of first actuator 108 and second actuator 109 may be coupled to one or more control wires 105, and each of the one or more control wires 105 may extend from handle body 107 through shaft 104 to distal portion 102.

Shaft 104 may be cylindrical and may include a central lumen extending longitudinally through shaft 104. Portions of shaft 104 have been removed from the view of medical device 100 shown in FIG. 1 for illustration purposes only; a proximal end of shaft 104 may be coupled to handle body 107 and a distal end of shaft 104 may be coupled to distal component 124. Shaft may comprise any suitable flexible material to facilitate insertion into and navigation through a body lumen.

Distal portion 102 may include distal component 124, first jaw member 125, second jaw member 126, needle 130, first pivot component 132, second pivot component 133, and first pivot rod 127. Distal component 124 may include a central lumen extending longitudinally through distal component 124, and may be configured to receive portions of first jaw member 125, second jaw member 126, first pivot component 132, and second pivot component 133. First pivot rod 127 may be coupled to a distal end portion of distal component 124. Distal component 124 may include a first protrusion 145 and a second protrusion 146 (shown in FIG. 5) that form a U-shaped distal end of distal component 124, and form a three-sided opening 142 of distal component 124. Three-sided opening 142 may allow needle 130 to travel proximally and distally through distal component 124, for example from (i) a first position entirely within distal component 124 positioning the distalmost end of needle 130 proximal from a distalmost end of distal component 124 to (ii) a second position in which the distalmost end of needle 130 is positioned distal from the distalmost end of distal component 124.

Each of first jaw member 125 and second jaw member 126 may be partially ellipsoidal in shape, with respective recessed portions 251, 252 and curved radially-outer surfaces 171, 172. First jaw member 125 may have a first opening 173 into recessed portion 252, and first opening 173 may be oval shaped. Second jaw member 126 may have a second opening 174 into recessed portion 251, and second opening 174 may be oval shaped. In other examples, first opening 173 and second opening 174 may be circular, rectangular, triangular, pentagonal, hexagonal, or any other suitable shape. First opening 173 and second opening 174 may have substantially identical or otherwise complementary shapes such that first opening 173 aligns with second opening 174 when first jaw member 125 and second jaw member 126 are in a closed configuration (shown in FIG. 2B). Each of first opening 173 and second opening 174 may include a recessed portion 555, 556 (shown in FIG. 5), respectively, at a proximal end of the opening. When jaw members 125,126 are in a closed position, these recessed portions 555, 556 may form a proximal-facing opening configured to receive needle 130 and allow needle 130 to move proximally and distally through the proximal-facing opening.

A plurality of apertures 241 may extend through first jaw member 125 and second jaw member 126. In some examples, each aperture 241 may have a length and/or width (e.g., a diameter in the case of circular apertures) in the range of 1 mm to 3 mm. Each aperture 241 may extend through the radially-outer surfaces 171, 172 into recessed portions 252, 251. In some examples, for each of the first jaw member 125 and the second jaw member 126, recessed portions 251, 252 and radially-outer surfaces 171, 172 may form respective basket portions 176, 177. The basket portions 176, 177 may comprise a flexible metallic mesh, such as a metallic mesh comprising stainless steel or Nitinol. In other examples, basket portions 176, 177 may comprise any other biocompatible metal or non-metal material(s). In some examples, basket portions 176, 177 may comprise a material resistant to electrification, e.g., to prevent electrification from needle 130 from spreading outside of the basket portions 176, 177. In some examples, basket portions 176, 177 may include a polymer coating, e.g., coated with polytetrafluoroethylene (PTFE), to facilitate trapping dust particles in jaw members 125, 126 during a procedure.

In some examples, basket portions 176, 177 may be flexible such that the sizes of recesses 252, 251 may be increased when basket portions 176, 177 are stretched. The flexibility of first jaw member 125 and second jaw member 126 may allow basket portions 176, 177 to be wound together to allow first jaw member 125 and second jaw member 126 to fit within a working channel of a medical device, such as an endoscope or a ureteroscope. FIG. 4 illustrates first jaw member 125 and second jaw member 126 wound around each other (e.g. folded around each other, etc.), thus decreasing the radial diameter of first jaw member 125 and second jaw member 126. Each of first jaw member 125 and second jaw member 126 may be biased towards an expanded configuration shown in FIGS. 1, 2A and 2B, and thus first jaw member 125 and second jaw member 126 may be configured to expand radially-outward, relative to central longitudinal axis 199, when first jaw member 125 and second jaw member 126 are pushed distally out of a working channel of a medical device, such as an endoscope or a ureteroscope.

In some examples, basket portions 176, 177 may have a flexible net structure similar to a fishing net design. FIG. 3 illustrates an exemplary medical device 300 with a basket portion 302 of a snare loop 301 coupled to a shaft 303. Any of the attributes shown in FIG. 3, particularly of basket portion 302, may be incorporated into medical device 100, and specifically into first jaw member 125 and second jaw member 126. Basket portion 302 may include a plurality of strands, such as interwoven strands of a biocompatible material, the strands being interlaced to form a net structure and/or bonded together via one or more adhesives. In some examples, basket portion 302 may be bonded or otherwise adhered to a loop member 306 extending distally from proximal shaft 303, and loop member 306 may be oval-shaped and may be coated in PTFE.

Referring again to FIG. 2A, first jaw member 125 and second jaw member 126 may include respective first and second pivot shafts 182, 183 extending proximally from basket portions 176, 177. The first and second pivot shafts 182, 183 may be rectangular, cylindrical, or any other suitable shape. In some examples, the first pivot shaft 182 of first jaw member 125 and the second pivot shaft 183 of second jaw member 126 may each be coupled to first pivot rod 127, and first pivot shaft 182 and second pivot shaft 183 may rotate about first pivot rod 127. In some examples, a proximal end of first pivot shaft 182 may be coupled to first pivot component 132, and a proximal end of second pivot shaft 183 may be coupled to second pivot component 133. First pivot component 132 and second pivot component 133 may each be coupled to one or more other pivot components and to a control wire extending through shaft 104 to handle assembly 106. The control wire coupled to the first jaw member 125 and the second jaw member 126 may extend longitudinally through shaft 104 and may be coupled to first actuator 108 of handle assembly 106.

FIG. 5 illustrates first jaw member 125 and second jaw member 126 in an open configuration, with needle 130 positioned between first jaw member 125 and second jaw member 126. Needle 130 may be cylindrical and may include a central lumen 731 extending longitudinally through needle 130 to an opening 732 at a distal end of needle 130. Lumen 731 may be configured to receive fluid and/or suction from a proximal end of medical device 100, and may be used to aspirate material from a target site and/or provide fluid irrigation to a target site. In some examples, lumen 731 may be configured to receive a tissue sample during a procedure. Needle 130 may be removable from medical device 100 for processing of the tissue sample. For example, the needle 103 may be provided to a pathology lab for further study. In some examples, needle 130 does not include a lumen. In such cases, needle 130 may be tapered to a sharp distalmost end 733 configured to cut tissue. Needle 130 may extend longitudinally entirely or partially through distal component 124, and/or may extend partially or entirely through shaft 104.

As shown in FIG. 5, needle 130 may extend longitudinally through shaft 104 and through a central longitudinal axis 199 of distal component 124. First pivot component 132 and first pivot shaft 182 may be positioned adjacent to needle 130 and may be entirely spaced from central longitudinal axis 199. Second pivot component 133 and second pivot shaft 183 may be positioned adjacent to needle 130 on an opposite side of needle 130 as first pivot component 132 and first pivot shaft 182 (e.g. shown in FIG. 5), and may be entirely spaced from central longitudinal axis 199. Proximal recessed portions 555, 556 of each of first jaw member 125 and second jaw member 126, respectively, may each include a curved edge configured to extend circumferentially around needle 130 when first jaw member 125 and second jaw member 126 are in a closed position.

Needle 130 may be coupled to second actuator 109 of handle assembly 106. In some examples, a control wire may be coupled to needle 130 and extend partially or entirely through shaft 104 to second actuator 109. Second actuator 109 may be configured to control proximal and distal movement of needle 130, and a user may move needle 130 proximally and distally to cut and/or otherwise manipulate tissue. In some examples, needle 130 may be conductive and configured to receive electrical energy from an energy source, e.g., needle 130 being configured to cut tissue or other material and/or cauterize tissue via electrocautery. Needle 130 may be sized to fit between first jaw member 124 and second jaw member 126, and may be configured to be received within first basket portion 176 and second basket portion 177 when first jaw member 124 and second jaw member 125 are in a closed configuration.

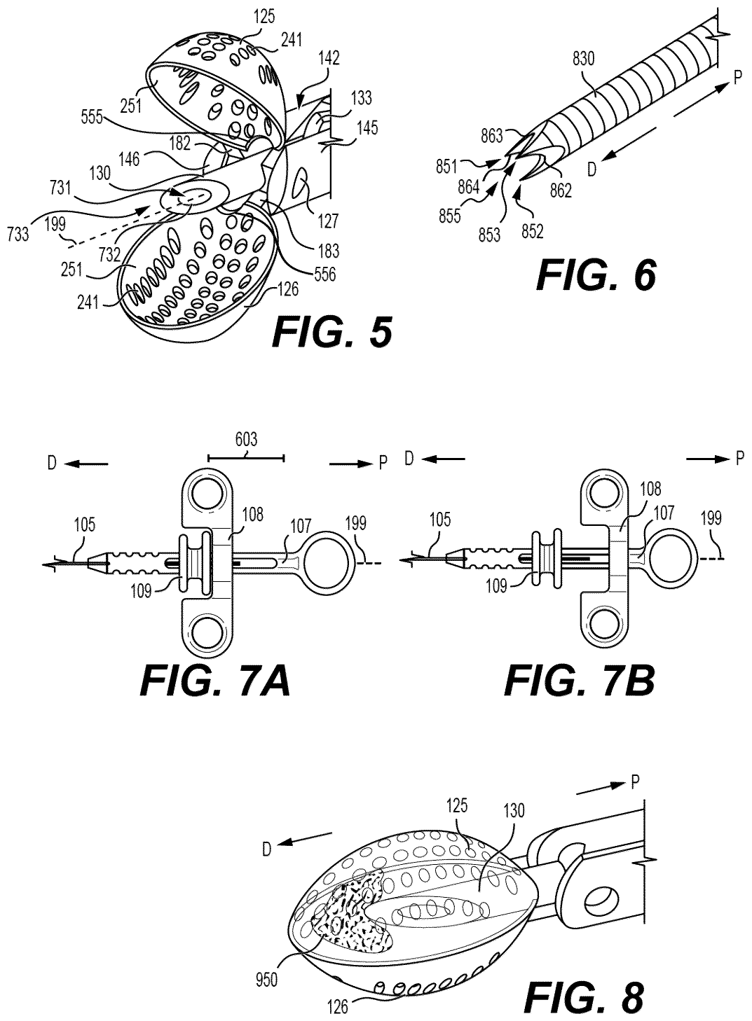

FIG. 6 illustrates another exemplary needle 830 that may be included in any of the medical devices 100, 1000 described herein, and may have any of the features described in relation to other needles 130, 1130. Needle 830 may be cylindrical and may include a central longitudinal lumen 831 extending the entire length of needle 830. Needle 830 may include three sharp points 852, 853, 854 at a distalmost end of needle 830. The three sharp points 852, 853, 854 may be evenly spaced circumferentially around a distalmost end of needle 830. In some examples, sharp point 852 may be connected to sharp point 853 via a curved sharp edge 862, sharp point 853 may be connected to sharp point 854 via a curved sharp edge 863, and sharp point 854 may be connected to sharp point 852 via a curved sharp edge 864. In other examples, needle 830 may have one, two, four, five, six, or any other suitable number of sharp points 852, 853, 854 and/or curved sharp edges 862, 863, 864. Needle 830 may be configured to receive and conduct electrical energy to cut and/or ablate and/or cauterize material, such as tissue.

In operation during a medical procedure, a user may position medical device 100 at a target site within a body of a patient. For example, a user may insert medical device 100 into a working channel of a ureteroscope or other medical device and move a distal end of the ureteroscope or other medical device to the target site. The user may then deploy medical device 100 by pushing medical device 100 distally such that jaw members 125, 126 extend distally beyond a distal end of the ureteroscope or other medical device. Then, in some examples, the user may transition jaw members 125, 126 from a closed configuration (e.g. FIG. 2B) to an open configuration (e.g. FIG. 2A) by moving first actuator 108 distally relative to handle body 107 to the handle position shown in FIG. 7A. The handle position shown in FIG. 7A may be referred to as the open handle position corresponding to when jaw members 125, 126 are in an open configuration. The user may then position jaw members 125, 126 proximate to material that the user would like to break apart and/or remove from the target site. The user may then transition jaw members 125, 126 from the open configuration to a closed configuration by pulling first actuator 108 proximally to the closed handle configuration shown in FIG. 7B, which moves jaw members 125, 126 to a closed configuration and may trap material at the target site within basket portions 176, 177.

FIG. 8 illustrates jaw members 125, 126 in a closed configuration with material 950 trapped within jaw members 125, 126, and needle 130 deployed within jaw members 125, 126. The user may actuate needle 130 while jaw members 125, 126 are in a closed configuration, for example by moving second actuator 109 proximally or distally relative to handle body 107. For example, while the handle is in a closed handle configuration shown in FIG. 7B, the user may move second actuator 109 proximally and/or distally to pierce and/or break apart material 950 trapped between jaw members 125, 126. In some examples, the user may apply electrical energy to needle 130, via one or more actuators of medical device 100, to cut and/or ablate material 950 while material 950 is trapped between jaw members 125, 126. Jaw members 125, 126 may be configured to protect anatomy of the patient at the target site proximate of jaw members 125, 126 while the user actuates needle 130 and applies electrical energy to needle 130. Additionally or alternatively, the user may apply suction through needle 130 to collect material 950 within lumen 731, for example for collection of a biopsy and/or to remove material 950 from the patient. In some examples, material 950 may include one or more stones.

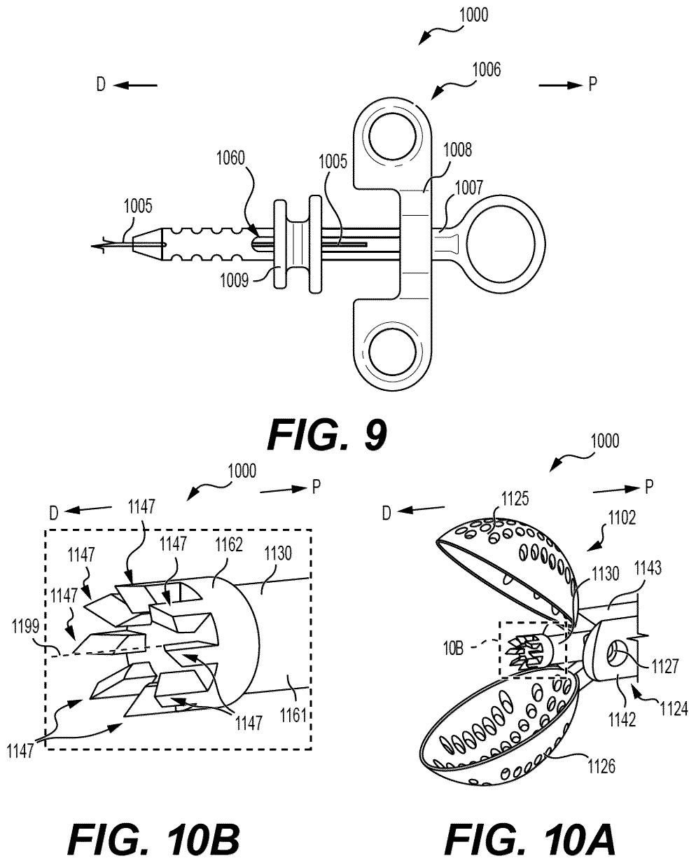

FIGS. 9, 10A, and 10B illustrate another exemplary medical device 1000 according to aspects of the present disclosure. Medical device 1000 may have any of the features described herein in relation to medical device 100. FIG. 9 illustrates a handle assembly 1006 of medical device 1000, which may include handle body 1007, first actuator 1008, and second actuator 1009, and may have any of the features described hereinabove in relation to handle assembly 106. Handle assembly 1006 may be coupled to a shaft 1005, and shaft 1005 may extend to a distal portion 1102 of medical device 1000. FIG. 10A illustrates distal portion 1102 of medical device 1000. Distal portion 1102 may include first jaw member 1125, second jaw member 1126, needle 1130, and a distal component 1124. Distal component 1124 may include a first protrusion 1142 and a second protrusion 1143 that form a U-shape, and a pivot rod 1127 may extend between the first protrusion 1142 and the second protrusion 1143. In some examples, medical device 1000 does not include pivot rod 1127.

Referring to FIG. 9, handle assembly 1006 may include a motor 1060 configured to rotate a control wire 1005 coupled to needle 1130. Motor 1060 may be configured to rotate control wire 1005 about a longitudinal axis of control wire 1005, such as the central longitudinal axis of control wire 1005. In some examples, motor 1060 may be operably coupled to an actuator of handle assembly 1006, such as a button, switch, or other actuator, to control motor 1060. Additionally or alternatively, motor 1060 may be configured for external or remote control, e.g., via wired or wireless communication with a remote controller, a computer interface, or any other control unit in wired or wireless communication with motor 1060. In some examples, motor 1060 may be moveable in a proximal and a distal direction relative to handle body 1007, such as via movement of second actuator 1009. Motor 1060 may drive rotation of needle 1130 to facilitate breaking apart material within a patient during a medical procedure using medical device 1000.

FIG. 10B illustrates a magnified view of a distal portion of needle 1130. Needle 1130 may include an expanded distal portion 1162 that may have a larger diameter than a proximal portion 1161 of needle 1130. Distal portion 1162 may include a series of distal protrusions 1147 evenly spaced circumferentially around distal portion 1162, and each distal protrusion 1147 may extend distally substantially parallel to a central longitudinal axis 1199 of needle 1130. Each distal protrusion 1147 may include a sharp distalmost end configured to cut material, such as tissue within a patient. Each distal protrusion 1147 may include an angled distal-facing surface that is substantially the same angle as each other distal-facing surface of each other distal protrusion 1147. These angled distal-facing surfaces of distal protrusions 1147 may be configured to facilitate cutting tissue by rotating needle 1130 about central longitudinal axis 1199.

During operation of medical device 1000, a user may first capture material between first jaw member 1125 and second jaw member 1126 by actuating first actuator 1008. The user may then move needle 1130 distally via second actuator 1009 to engage material trapped between first jaw member 1125 and second jaw member 1126. The user may then actuate motor 1060 to initiate rotation of needle 1130 about its longitudinal axis 1199, to facilitate breaking apart the material trapped between first jaw member 1125 and second jaw member 1126.

In some examples, medical device 1000 does not include motor 1060. In such cases, the user may rotate second actuator 1009 about a longitudinal axis of medical device 1000 relative to handle body 1007 to rotate needle 1130. In some examples, handle assembly 1006 may include a gearing mechanism configured to increase the amount of rotation translated to needle 1130 via the rotation of second actuator 1009. For example, a single 360-degree rotation of second actuator 1009 may result in 720 degrees of rotation of needle 1130 due to a gearing mechanism incorporated into medical device 1000. In some examples, handle assembly 1006 does not include second actuator 1009. In such cases, needle 1130 may be longitudinally fixed to distal member 1124 such that needle 1130 can rotate about a longitudinal axis of needle 1130 and be prevented from moving in a proximal or distal direction relative to distal member 1124.

In any of the medical devices 100, 1000 described herein, heat may be applied to needle 130, 830, 1130, for example through application of a fluid (e.g., steam, heat-conductive gel, etc.) through a lumen 731 of the needle 130, 830, 1130. By applying heat to needle 130, 830, 1130, the needle 130, 830, 1130 may soften material at a target area within a patient to facilitate breaking the material apart or otherwise manipulating the material.

The medical devices, systems, and methods discussed in this disclosure may facilitate removal of kidney stones or other tissue from a patient's body, may increase the amount of material that can be removed from a patient during a single procedure, may reduce the procedure time, and/or may reduce the risk of injury to the patient. For example, the devices in this disclosure may help to limit or prevent unintentional damage to a patient during electrocautery, and may reduce the risk of unintentional exposure to an electrocautery needle by encapsulating the needle with jaw members (e.g., basket portions discussed above, etc.).

Any of the medical devices 100, 1000 described herein may be made of any suitable biocompatible material, such as plastic, PEBAX, or any other suitable material. In any of the above-described examples, medical devices 100, 1000 may include one or more components that are metallic, polymeric, machined, formed, stamped, insert molded, or any combination thereof. Any aspect(s) of any one of the above-described examples of medical device 100, 1000 may be incorporated into any of the other examples of medical devices 100, 1000 described herein.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A medical device comprising:
a handle assembly including a first actuator;
a shaft extending distally from the handle assembly;
a first jaw member movably coupled to a distal portion of the shaft;
a second jaw member movably coupled to the distal portion of the shaft and to the first jaw member; and
a needle positioned between the first jaw member and the second jaw member;
wherein the needle is configured to receive electrical energy to facilitate cutting, ablating, and/or cauterizing tissue.

2. The medical device of claim 1, wherein the first jaw member includes a first basket portion including a first recessed portion; and wherein the second jaw member includes a second basket portion including a second recessed portion.

3. The medical device of claim 2, wherein each of the first basket portion and the second basket portion includes a plurality of apertures.

4. The medical device of claim 2, wherein each of the first basket portion and the second basket portion includes a polymer coating.

5. The medical device of claim 2, wherein the first actuator is configured to transition the first jaw member and the second jaw member between an open configuration in which the first jaw member and the second jaw member are spaced from each other and a closed configuration in which the first basket portion and the second basket portion form an enclosure.

6. The medical device of claim 1, wherein the handle assembly further comprises a second actuator configured to move the needle relative to the first jaw member and the second jaw member.

7. The medical device of claim 6, wherein the first actuator includes a recess configured to receive the second actuator.

8. The medical device of claim 1, wherein the needle includes a lumen configured to receive a fluid.

9. The medical device of claim 1, wherein the needle is electrically conductive.

10. The medical device of claim 1, wherein a distalmost portion of the needle includes a plurality of sharp tips.

11. The medical device of claim 1, wherein the handle assembly further comprises a motor configured to rotate the needle about a longitudinal axis of the needle.

12. The medical device of claim 2, wherein the first basket portion and the second basket portion are flexible.

13. The medical device of claim 2, wherein the first basket portion and the second basket portion are heat resistant.

14. The medical device of claim 2, wherein the first basket portion includes a first oval opening and the second basket portion includes a second oval opening, wherein the first oval opening and the second oval opening are substantially the same size and configured to align with each other when the first jaw member and the second jaw member are in a closed configuration.

15. The medical device of claim 1, wherein, when the first jaw member and the second jaw member are in a closed configuration that forms an enclosure, the first basket portion and the second basket portion form a proximal opening configured to receive the needle.

16. A medical device comprising:
a handle assembly including a first actuator and a second actuator;
a shaft extending distally from the handle assembly;
a first jaw member movably coupled to a distal portion of the shaft;
a second jaw member movably coupled to the distal portion of the shaft; and
a needle positioned between the first jaw member and the second jaw member;
wherein the first actuator is configured to actuate the first jaw member and the second jaw member to transition between an open configuration and a closed configuration; and
wherein the second actuator is configured to move needle relative to the first jaw member and the second jaw member.

17. The medical device of claim 16, wherein the needle is electrically conductive.

18. The medical device of claim 17, wherein the needle includes a lumen configured to receive a fluid.

19. The medical device of claim 16, wherein, when the first jaw member includes a first basket portion and the second jaw member includes a second basket portion, and wherein the first jaw member and the second jaw member are in the closed configuration, the first basket portion and the second basket portion form a proximal opening configured to receive the needle.

20. A method comprising:
positioning a shaft of a medical device at target site of a patient;
moving a first jaw member and a second jaw member of the medical device from an open configuration in which the first jaw member and the second jaw member are spaced from each other to a closed configuration in which the first jaw member and the second jaw member form an enclosure;
moving a needle of the medical device distally into the enclosure; and
applying electrical energy to the needle.
